SEQUENCE LISTING (16 sequences)

SEQ ID NO: 1 - DNA, 210 bp, Wolinella succinogenes
atgaatctcatcatcaacggagagaataagagttttgaaaaagagggcttaagcgtgaaggagcttttggttttagaatcggttaaaatgcctgaaatggtctccattcagctcaatgacgagtttttaagagagcctgagtatgcgaccacttcgcttaaagagggcgatacgattaactttttatatttcatgggagggggcgcatga

SEQ ID NO: 2 - protein, 69 aa, Wolinella succinogenes
MNLIINGENKSFEKEGLSVKELLVLESVKMPEMVSIQLNDEFLREPEYATTSLKEGDTINFLYFMGGGA

SEQ ID NO: 3 - DNA, 393 bp, Wolinella succinogenes
atgctcaaaatccctaaagcgctctttgattccattatcgagcacgcccaaagagagcttcccttagaggcgtgcggctatgtagctggagtggagggcgaggtgaagcggctctttcctatgaggaatgtcgatgcgagccctgagcatttcagctttgatcccgccgaacaatttagcgcttttaaagaggcgcaaaaagagggattgcggctcattggctgctatcactctcacccaagcactcctgcaagaccctccgatgaggatattcgcctagcctatgacagtagcctcagctacctcattgtctcgctggccaaggagcctgttttaaactcttttaaaatcaaagagggagtcgtcactcccgaaaatatcgaggtgatctaa

SEQ ID NO: 4 - protein, 130 aa, Wolinella succinogenes
MLKIPKALFDSIIEHAQRELPLEACGYVAGVEGEVKRLFPMRNVDASPEHFSFDPAEQFSAFKEAQKEGLRLIGCYHSHPSTPARPSDEDIRLAYDSSLSYLIVSLAKEPVLNSFKIKEGVVTPENIEVI

SEQ ID NO: 5 - DNA, 819 bp, Wolinella succinogenes
atgagagagtttagcgaagaggagctagaacgctactcaaggcatatcatccttgaagaggtgggaatcgaggggcaagagaagatcatgaactccaaagtccttatcatcggtgcaggcggacttggttcgcccattgccttctatttggccgcagcgggagtgggagagatcggaatcatcgatggggatgtggtggatcggagcaatcttcagcgccagatcattcacaccacggatgagattggaatccccaaagtcgaatcggcacgccgcaagctcaaggcgctcaatcccaatattcgagttcaaacttggcagatcatgatcaatgccgaaaacatctcacgaatcatcgccccctacgattttatcatcgatgggacggataattttgcggccaaattcctcatcaatgatgcgtgtgtcatggcgggcaaaccctactcccacggtggaatcttgaaatttgcagggcagagcatgaccataaaaccgggagagagcgcctgctatgcgtgtgtctttgatcagcctccccccgctggctctattcccacctgctctagcgcagggattttaggggcgattgcagggatgcttggcaccattcaagccgccgaggcgctcaaagtgattacaggcgtaggcgaacccctctataatcgccttttaagctttgatgccaaaagcatgaatttccgcaccgtgaaattcaccaaaaacccccattgtcgtgtttgcggaggcgagggagtgaagattttacgcgaatatgaacaacccatatgcgaggtgaatcatgctcaaaatccctaa

SEQ ID NO: 6 - protein, 272 aa, Wolinella succinogenes
MREFSEEELERYSRHIILEEVGIEGQEKIMNSKVLIIGAGGLGSPIAFYLAAAGVGEIGIIDGDVVDRSNLQRQIIHTTDEIGIPKVESARRKLKALNPNIRVQTWQIMINAENISRIIAPYDFIIDGTDNFAAKFLINDACVMAGKPYSHGGILKFAGQSMTIKPGESACYACVFDQPPPAGSIPTCSSAGILGAIAGMLGTIQAAEALKVITGVGEPLYNRLLSFDAKSMNFRTVKFTKNPHCRVCGGEGVKILREYEQPICEVNHAQNP

SEQ ID NO: 7 - protein, 764 aa, Wolinella succinogenes
MSHYTLPPSVAEDFASFKESHADFMAGKLDALTFKTIRVPFGIYEQRESDTYMVRVKLAGGILTPAQLHSLALLAEHYAKPHLHVTTRGGVQLHYAKLQDLPQIIQALHEMGLTGRGGGGNTVRNITADPYAGIAPDEAFDVTPCALSLTTKMLETKDSYSLPRKFKIAFSGSSEDRGGATYIDVGFIAKIHEGVRGFRLFVAGGMGAKSRLGSAFIDFLPQEEIFLFSQAIKQVFDQHGNRKNKHAARLRFLIEELGEGQFRELVFKEVQALRDQGGWEIKLEEAFEAIPLESDEIPPLNQEQKLWWNRFVIPQKQKGYYSAKVPLKLGDLESEHAKSLAQALQGFKYGKESIRFGSDQNLYLRNLQADELLSLYPLIQELSGQSSRARILGDMVACTGAATCQLGITRPRGAVVAIEKELQKANIDLDALQGFRIHLSGCPNSCGKHAIADLGFFGKVNREGGHPYPAYNVLVGAIIQEDSTRFAKKIAEVSAYALPLFVTEVLKLWLHAKPHYANFAAWVDQEGEAQIIHLASSYAKIPSFEEDKNPYFDYGSEEIFSLKGRGVGECSAGMYDLIEADKKALKEALEGGDEEENLAKIRLLASRMLLITKGEEARDERSVLQAFRRLFVEGGLIDSSFAPLLEGRPRIELKALGEAVIALYGTMDNSLKFAKEQPKEAPLAPTSSSSSAHRFKDYQGVACPMNFVKTKMDLAQMQSGEILEILLDEGAPIENVPKSVANEGHLILGQTKEGKGWRVRIQKR

SEQ ID NO: 8 - DNA, 2295 bp, Wolinella succinogenes
atgagccactacaccctacccccctccgtcgctgaggattttgcctctttcaaagagagtcacgccgactttatggcgggcaaactcgatgcactgacctttaaaaccattcgtgttccctttggaatctatgagcaacgagagagcgacacctatatggtgagagtgaagctagcagggggcattcttactcccgctcaactccactcgctcgcccttctagccgagcattacgccaaaccccatctccatgtcaccacgcgcggaggcgtccagctccactatgccaagctccaagatctcccccaaatcatccaagcgcttcatgagatggggctcacagggcgaggcggaggcgggaacaccgtgcgcaacatcactgccgatccctatgcaggaatcgcccctgatgaggcatttgatgtcactccttgtgcgctctctctcaccacgaaaatgcttgagacaaaagactcctattcgctcccaagaaaattcaaaatcgccttcagtggctctagcgaggataggggcggagccacctacatcgatgtgggattcatcgccaagatccacgaaggagtgcgaggattccgtctatttgtcgctgggggcatgggggctaaatcacgccttggaagcgcttttatcgactttttaccccaagaagagatattccttttttctcaagccatcaagcaggtttttgaccagcacggcaatcgcaaaaacaagcacgccgcaagacttcgattcctcatcgaggagcttggagaggggcaatttagagagcttgtctttaaagaggtccaagcgcttcgcgatcaaggggggtgggagattaagctagaggaggcttttgaggcaatccctttagagagcgatgagattcctccattaaatcaagagcaaaaactctggtggaatcgctttgttatccctcaaaaacaaaaaggctactacagtgccaaagtccccctgaaattgggcgacttggaatcagaacacgccaaatcccttgcccaagcccttcaaggattcaagtatggcaaagagtcgattcgttttggaagcgatcagaatctctacctaagaaacctccaagccgatgaacttctctcgctctacccccttattcaagagctctcaggacaatcaagccgcgcaaggattcttggggatatggtcgcttgcacaggcgcggccacttgccagcttggaatcacgcgccccagaggagccgtggtggcaatagaaaaagagctccaaaaagccaatatcgacctagatgcgcttcaaggctttagaatccatctctctggatgccccaatagctgcggaaaacacgccatcgctgatttgggattttttggcaaagtgaatcgagagggaggtcatccctatcccgcctataatgtccttgtgggcgccatcatccaagaagattccactcgatttgccaaaaaaatcgccgaggtgagcgcctatgctcttcctctttttgtgacagaggtgctcaagctttggctccatgccaagccccattatgcgaactttgctgcatgggtggatcaagagggcgaggctcaaatcatccacctcgcctcttcctatgccaagattcctagctttgaagaggataaaaacccctactttgactacggaagcgaagagatattttcgctcaaaggtcgaggagtgggtgagtgcagcgcgggaatgtatgacctaattgaggcggataaaaaagcactcaaagaggcgctagagggaggagacgaggaggagaatctcgccaaaatccgcctgctcgcctcaagaatgctcctcatcaccaaaggcgaagaggcgcgcgatgaacgctcggtgcttcaggcgttcaggcgactttttgtcgaaggaggattgattgattcctcttttgcgccccttttagaaggcaggccaagaatcgagctcaaagctctaggtgaggcggtcattgctctttatggcaccatggataatagcctgaaatttgccaaagagcagccaaaagaagcacctcttgcccccacctctagctcctctagtgcccatcgctttaaagattaccaaggggtcgcctgccccatgaactttgtcaagaccaaaatggacttagcccaaatgcaaagcggcgagattttggagattttgctcgatgagggcgctcccatcgaaaatgtccccaaatccgtagccaacgagggtcacctcatcctaggccaaaccaaagagggaaaagggtggcgcgtgaggattcaaaaacgatga

SEQ ID NO: 9 - DNA, 1140 bp, Leptospira meyeri
atgcctacctccgaacagaacgagttttcccacggatccgtaggtgtcgtatatactcagagcattcgatttgagtctttgactctagaggggggtgaaaccatcactcctcttgaaattgcctacgaaacgtatggcactctcaatgaaaaaaaagacaatgccattctagtttgccatgcgctttcgggagatgctcatgcagcaggtttccatgaaggagacaaacgtcctggctggtgggattattatattggaccgggcaaatcctttgataccaatcgttactttatcatttcttccaacgtaattggtggttgtaagggttccagtggaccacttaccatcaatgggaaaaatggaaaaccattccaatccacttttccctttgtctccataggagatatggtgaatgctcaagaaaaattaatcagccattttggaattcataaactatttgctgttgccggtggttcgatgggtggaatgcaagccttacaatggtcagtcgcatacccagatcggctcaaaaattgtatcgtgatggcatcttcttccgaacattctgcacaacaaattgcctttaatgaagtgggaagacaagccattctttctgatcccaattggaaccaaggtttgtacacccaggaaaacagaccgtcaaagggacttgctcttgctcgaatgatgggtcatatcacttacttaagcgatgaaatgatgagagaaaaatttggtcgtaaaccacccaaaggaaatatccaatccacagactttgcggtaggaagttatctaatctaccaaggcgaatcctttgtcgatcggtttgatgcaaactcatatatttatgttacaaaagcattggatcattttagtttaggtacaggaaaagaacttacaaaggtattggcaaaagtgagatgccggtttttggtagtggcttatacttccgattggttgtatccaccgtatcaatctgaagaaattgtgaaatctttggaagtgaatgctgttcccgttagttttgtagaactcaacaatccagcaggacgacatgatagttttttgttaccaagtgagcaacaagactcgatcctaagagattttttaagttctacggacgaaggagttttcctttga

SEQ ID NO: 10 - protein, 378 aa, Leptospira meyeri
MPTSEQNEFSHGSVGVVYTQSIRFESLTLEGGETITPLEIAYETYGTLNEKKDNAILVCHALSGDAHAAGFHEGDKRPGWWDYYIGPGKSFDTNRYFIISSNVIGGCKGSSGPLTINGKNGKPFQSTFPFVSIGDMVNAQEKLISHFGIHKLFAVAGGSMGGMQALQWSVAYPDRLKNCIVMASSSEHSAQQIAFNEVGRQAILSDPNWNQGLYTQENRPSKGLALARMMGHITYLSDEMMREKFGRKPPKGNIQSTDFAVGSYLIYQGESFVDRFDANSYIYVTKALDHFSLGTGKELTKVLAKVRCRFLVVAYTSDWLYPPYQSEEIVKSLEVNAVPVSFVELNNPAGHDSFLLPSEQQDSILRDFLSSTDEGVFL

SEQ ID NO: 11 - DNA, 1224 bp, Wolinella succinogenes
gtgaggggattcaccacgagggcgcttcatgttcccaaggccaagagagacgtccatggagcgcttcgcacgcccgtctatgataacgcggcgtttgagtttgaaaatagcgatgagatcgcccaagtttccttgggtagggcacttgggcatgtctatagccgctctagcaaccccacggtggaggatttggagcagcgcctcaagaatctcacgggagccttgggggtgttggcgctagggagtgggatggcagcgatctcaacggcgattttgaccttggcgagggcaggggatagtgtggtcaccaccgatcgtctctttgggcacaccctctcgctctttcaaaagacactgcctagctttggaatcgaggttcgttttgtggatgtgatggattctctagcagtggagcatgcctgtgatgagacaaccaagcttcttttcttggagaccatcagcaatcctcaacttcaagtggccgatctagaggctctctctaaagtggtgcacgccaaaggaatcccactagtggtcgatacgaccatgacccctccctatcttttggaggcaaaacgcttaggggtggacatcgaagtgctctcttcaaccaaattcatctcaggcggaggaacaagcgtgggaggagtcttgattgatcatggactttttgagtggaagagtctcccttcgctcgctccctattatgccaaggcggggccgatggctttcctctacaaggcgcgcaaggaggtgtttcaaaatctaggaccctcgcttagcccccacaacgcctatcttcaaagtttggggttggagacgatggcgcttcgaatcgagcgttcgtgtcaaaacgcccaagagcttgcgcattggcttttgtctatccctcaggtgaaatgcgtcaatcacccttcgctccctgattctcctttttatgcgattgctaagcgtcagtttcgctacgcaggctcgattctcacctttgagttggagagcaaggaggcctcctatcgcttcatggatgcgctcaagctcattcggcgcgccaccaatatccatgacaataaaagcctcatcctctccccctatcatgtcatctatgccctcaatagccacgaagagcgcttaaagcttgagatatctcctgcaatgatgaggctttctgtgggaattgaagagattgaagatctgaaagaggatattttgcaagcgctatgttaa

SEQ ID NO: 12 - protein, 407 aa, Wolinella succinogenes
VRGFTTRALHVPKAKRDVHGALRTPVYDNAAFEFENSDEIAQVSLGRALGHVYSRSSNPTVEDLEQRLKNLTGALGVLALGSGMAAISTAILTLARAGDSVVTTDRLFGHTLSLFQKTLPSFGIEVRFVDVMDSLAVEHACDETTKLLFLETISNPQLQVADLEALSKVVHAKGIPLVVDTTMTPPYLLEAKRLGVDIEVLSSTKFISGGGTSVGGVLIDHGLFEWKSLPSLAPYYAKAGPMAFLYKARKEVFQNLGPSLSPHNAYLQSLGLETMALRIERSCQNAQELAHWLLSIPQVKCVNHPSLPDSPFYAIAKRQFRYAGSILTFELESKEASYRFMDALKLIRRATNIHDNKSLILSPYHVIYALNSHEERLKLEISPAMMRLSVGIEEIEDLKEDILQALC

SEQ ID NO: 13 - DNA, 1224 bp, Wolinella succinogenes
atgaggggattcaccacgagggcgcttcatgttcccaaggccaagagagacgtccatggagcgcttcgcacgcccgtctatgataacgcggcgtttgagtttgaaaatagcgatgagatcgcccaagtttccttgggtagggcacttgggcatgtctatagccgctctagcaaccccacggtggaggatttggagcagcgcctcaagaatctcacgggagccttgggggtgttggcgctagggagtgggatggcagcgatctcaacggcgattttgaccttggcgagggcaggggatagtgtggtcaccaccgatcgtctctttgggcacaccctctcgctctttcaaaagacactgcctagctttggaatcgaggttcgttttgtggatgtgatggattctctagcagtggagcatgcctgtgatgagacaaccaagcttcttttcttggagaccatcagcaatcctcaacttcaagtggccgatctagaggctctctctaaagtggtgcacgccaaaggaatcccactagtggtcgatacgaccatgacccctccctatcttttggaggcaaaacgcttaggggtggacatcgaagtgctctcttcaaccaaattcatctcaggcggaggaacaagcgtgggaggagtcttgattgatcatggactttttgagtggaagagtctcccttcgctcgctccctattatgccaaggcggggccgatggctttcctctacaaggcgcgcaaggaggtgtttcaaaatctaggaccctcgcttagcccccacaacgcctatcttcaaagtttggggttggagacgatggcgcttcgaatcgagcgttcgtgtcaaaacgcccaagagcttgcgcattggcttttgtctatccctcaggtgaaatgcgtcaatcacccttcgctccctgattctcctttttatgcgattgctaagcgtcagtttcgctacgcaggctcgattctcacctttgagttggagagcaaggaggcctcctatcgcttcatggatgcgctcaagctcattcggcgcgccaccaatatccatgacaataaaagcctcatcctctccccctatcatgtcatctatgccctcaatagccacgaagagcgcttaaagcttgagatatctcctgcaatgatgaggctttctgtgggaattgaagagattgaagatctgaaagaggatattttgcaagcgctatgttaa

SEQ ID NO: 14 - protein, 407 aa, Wolinella succinogenes
MRGFTTRALHVPKAKRDVHGALRTPVYDNAAFEFENSDEIAQVSLGRALGHVYSRSSNPTVEDLEQRLKNLTGALGVLALGSGMAAISTAILTLARAGDSVVTTDRLFGHTLSLFQKTLPSFGIEVRFVDVMDSLAVEHACDETTKLLFLETISNPQLQVADLEALSKVVHAKGIPLVVDTTMTPPYLLEAKRLGVDIEVLSSTKFISGGGTSVGGVLIDHGLFEWKSLPSLAPYYAKAGPMAFLYKARKEVFQNLGPSLSPHNAYLQSLGLETMALRIERSCQNAQELAHWLLSIPQVKCVNHPSLPDSPFYAIAKRQFRYAGSILTFELESKEASYRFMDALKLIRRATNIHDNKSLILSPYHVIYALNSHEERLKLEISPAMMRLSVGIEEIEDLKEDILQALC

SEQ ID NO: 15 - DNA, 50 bp, artificial
  DmetA*11-1 homologous sequence for metA*11 deletion
tgtagtgaggtaatcaggttatgccgattcgtgtgccggacgagctaccc

SEQ ID NO: 16 - DNA, 50 bp, artificial
  DmetA*11-2 homologous sequence for metA*11 deletion
tcttctgtgatagtcgatcgttaagcgattcagcaccttacctcaggcac

(19) United States
(12) Patent Application Publication
(10) Pub. No.: US 2018/0223319 A1
(43) Pub. Date: Aug. 9, 2018

(54) PROTEIN THIOCARBOXYLATE-DEPENDENT L-METHIONINE PRODUCTION BY FERMENTATION

(71) Applicant: EVONIK DEGUSSA GMBH, Essen (DE)

(72) Inventors: Philippe SOUCAILLE, Deyme (FR); Perrine VASSEUR, Martres sur Morges (FR)

(73) Assignee: Evonik Degussa GmbH, Essen (DE)

(21) Appl. No.: 15/750,516

(22) PCT Filed: Aug. 7, 2015
(86) PCT No.: PCT/IB2015/001888

§ 371 Date: Feb. 6, 2018

(51) Int. Cl. C12P 13/12; C12N 9/10

(52) U.S. Cl. CPC C12P 13/12; C12N 9/1029; C12Y 205/01049; C12N 9/1085; C12Y 203/01031

(57) ABSTRACT

The present invention relates to a recombinant microorganism useful for the production of L-methionine and process for the preparation of L-methionine. The microorganism of the invention is modified in a way that the L-methionine production is improved by using a thiocarboxylated protein as sulfur donor and by expressing an enzyme having homoserine O-acetyltransferase activity without feedback inhibition by methionine and/or S-adenosylmethionine and an enzyme having O-acetylhomoserine sulfhydrylase activity.

### FIELD OF THE INVENTION

The present invention relates to a recombinant microorganism useful for the production of L-methionine and process for the preparation of L-methionine. The microorganism of the invention is modified in a way that the L-methionine production is improved by using a thiocarboxylated protein as sulfur donor and by overproducing an enzyme having homoserine O-acetyltransferase activity without feedback inhibition by methionine and/or S-adenosylmethionine and an enzyme having O-acetylhomoserine sulfhydrylase activity.

### PRIOR ART

Sulfur-containing compounds such as cysteine, homocysteine, methionine or S-adenosylmethionine are critical to cellular metabolism. In particular L-methionine, an essential amino acid, which cannot be synthesized by animals, plays an important role in many body functions. Most of the methionine produced industrially is widely used as an animal feed and food additive.

With the decreased use of animal-derived proteins as a result of BSE (bovine spongifor encephalopathy) and chicken flu, the demand for pure methionine has increased. Commonly, D,L-methionine is produced chemically from acrolein, methyl mercaptan and hydrogen cyanide. However, the racemic mixture does not perform as well as pure L-methionine (Saunderson, 1985). Additionally, although pure L-methionine can be produced from racemic methionine, for example, through the acylase treatment of N-acetyl-D,L-methionine, this dramatically increases production costs. Accordingly, the increasing demand for pure L-methionine coupled with environmental concerns render microbial production of methionine an attractive prospect.

Other important amino acids, such as lysine, threonine and tryptophan are produced via fermentation for use in animal feed. Therefore, these amino acids can be made using glucose and other renewable resources as starting materials. The production of L-methionine via fermentation has not been successful yet, but the development of the technology is on going.

Different approaches for the optimization of L-methionine production in microorganisms have been described previously (see, for example, patents or patent applications U.S. Pat. No. 7,790,424, U.S. Pat. No. 7,611,873, WO02/10209, WO2005/059093, WO2006/008097, WO2007/0770441, WO2009/043803 and WO2012/098042); however, industrial production of L-methionine from microorganisms requires further improvements.

In these approaches the production of L-methionine is described as using cysteine as sulfur donor, and so the cysteine biosynthetic pathway is optimized by overproducing the different proteins involved in:
(i) The assimilation of sulfate into sulfide, with sequentially the sulfate adenylyltransferase, the adenylylsulfate kinase, the sulfite reductase and the 3′-phospho-adenylylsulfate reductase encoded by the operons cysDNC and cysJIH,
(ii) The cysteine synthesis, by the serine acetyltransferase and the cysteine synthase encoded by cysE and cysM genes,
(iii) The transsulfuration that means incorporation of the sulfur from cysteine to succinyl-homoserine giving γ-cysthathionine and then homocysteine via the O-succinylhomoserine (thiol)-lyase and the cystathionine-β-lyase encoded by metB and metC genes respectively.
   However, the optimization of the cysteine biosynthetic pathway necessary for the production of L-methionine is difficult. In fact, the accumulation of cysteine is toxic for the microorganism and so rapidly degraded by cysteinases if it is not used, which means if the cysteine is not produced exactly at the appropriate moment during the L-methionine production process. Moreover, among the known cysteinases present in E. coli there is MetC whose production is unavoidable and necessarily increased in the methionine producer strain as MetC is responsible for the conversion of γ-cysthathionine into homocysteine (WO2005/111202) in the methionine biosynthetic pathway.

To by-pass these difficulties, Arkema and CJ Cheiljedang Corporation claim the production of L-methionine in 3 steps: two biosynthesis processes to produce in one hand the methionine precursor, the O-acetyl-L-homoserine or the O-succinyl-L-homoserine, and in another hand the enzyme responsible for the transformation of the precursor in methionine; MetY or MetZ, the O-acetylhomoserine- or the O-succinylhomoserine sulfhydrylases respectively. In a third step, they describe the enzymatic bioconversion of the methionine precursor into L-methionine by MetY or MetZ enzyme in the presence of methyl-mercaptan as sulfur donor (WO2008/013432). Indeed, with this technology it is not necessary to optimize the production of cysteine in the microorganism, as the methyl-mercaptan is the sulfur donor.

Another alternative to the use of cysteine is described by CJ Cheiljedang Corporation and Cargill in patent WO2008/127240 in which they claim microorganisms that produce methionine from exogenous genes coding for homocysteine synthase and so providing a direct sulfhydrylation pathway, which means incorporation of the sulfur directly from sulfide to acetyl-homoserine giving in one step homocysteine.

In the literature it is described that protein thiocarboxylates are members of a growing family of biosynthetic sulfide donors and are involved in a variety of biosynthetic pathways, including vitamin B1 (Taylor et al., 1998) and cysteine (Agren et al., 2008). Recently, a protein thiocarboxylate-dependent methionine biosynthetic pathway was identified in Wolinella succinogenes (Krishnamoorthy et al., 2011). In this pathway, (i) the enzymes involved in assimilation of sulfate into sulfide are alike to those of E. coli; CysDNC for sulfate adenylyltransferase and adenylylsulfate kinase, CysH for 3′-phospho-adenylylsulfate reductase and Sir for sulfite reductase equivalent to CysJI; (ii) then the carboxy terminal alanine of a novel sulfur transfer protein, HcyS-Ala is removed in a reaction catalysed by a metalloprotease, HcyD, giving HcyS; (iii) HcyF, an ATP-utilizing enzyme, catalyses the adenylation of HcyS; (iv) HcyS acyl-adenylate (HcyS-COOAMP) then undergoes nucleophilic substitution by bisulfide produced by Sir to give the HcyS thiocarboxylate (HcyS-COSH); (v) this adds to O-acetylhomoserine to give HcyS-homocysteine in a PLP-dependent reaction catalysed by MetY (O-acetylhomoserine-sulfhydrylase); (vi) HcyD mediated hydrolysis liberates homocysteine, (vii) a final methylation catalysed by MetE, the homocysteine transmethylase, completes the methionine biosynthesis (cf FIG. 1.).

Inventors have found surprisingly that expression of the genes coding for the protein thiocarboxylate dependent methionine biosynthetic pathway of Wolinella succinogenes in a genetically modified microorganism producing methionine improves the methionine production and overcomes the problem of the cysteine production pathway optimization.

### SUMMARY OF THE INVENTION

The invention relates to a recombinant microorganism which produces methionine by fermentation wherein said microorganism expresses functional genes encoding a thiocarboxylated protein, a polypeptide having an homoserine O-acetyltransferase activity without feedback inhibition by methionine and/or S-adenosylmethionine and a polypeptide having O-acetylhomoserine sulfhydrylase activity. Method for the fermentative production of methionine comprising culturing said recombinant microorganism in an appropriate culture medium and recovering methionine from the culture medium is also an object of the invention.

### DESCRIPTION OF THE DRAWINGS

FIG. 1: Comparative Methionine biosynthesis pathway in Escherichia coli and in Wolinella succinogenes with the protein-thiocarboxylate HcyS.

### DETAILED DESCRIPTION OF THE INVENTION

Before describing the present invention in detail, it is to be understood that this invention is not limited to particularly exemplified methods and may, of course, vary. It is also to be understood that the terminology used herein is for the purpose of describing particular embodiments of the invention only, and is not intended to be limiting, which will be limited only by the appended claims.

All publications, patents and patent applications cited herein, whether supra or infra, are hereby incorporated by reference in their entirety.

Furthermore, the practice of the present invention employs, unless otherwise indicated, conventional microbiological and molecular biological techniques within the skill of the art. Such techniques are well known to the skilled worker, and are explained fully in the literature.

It must be noted that as used herein and in the appended claims, the singular forms “a”, “an”, and “the” include plural reference unless the context clearly dictates otherwise. Thus, for example, a reference to “a microorganism” includes a plurality of such microorganisms, and a reference to “an endogenous gene” is a reference to one or more endogenous genes, and so forth. Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

In the claims that follow and in the consecutive description of the invention, except where the context requires otherwise due to express language or necessary implication, the word “comprise”, “contain”, “involve” or “include” or variations such as “comprises”, “comprising”, “containing”, “involved”, “includes”, “including” are used in an inclusive sense, i.e. to specify the presence of the stated features but not to preclude the presence or addition of further features in various embodiments of the invention.

The term “methionine” and “L-methionine” designate the essential sulfur-containing amino-acid with chemical formula HO₂CCH(NH₂)CH₂CH₂SCH₃ and CAS number 59-51-8 or 63-68-3 for the specific L-isomer.

The term “microorganism”, as used herein, refers to a living microscopic organism, which may be a single cell, or a multicellular organism and which can generally be found in nature. In the context of the present invention, the microorganism is preferably a bacterium, yeast or fungus. More preferably, the microorganism of the invention is selected among Enterobacteriaceae, Bacillaceae, Streptomycetaceae, Corynebacteriaceae and yeast. Even more preferably, the microorganism of the invention is a species of Escherichia, Klebsiella, Thermoanaerobacterium, Corynebacterium or Saccharomyces. Yet, even more preferably, the microorganism of the invention is selected from Escherichia coli, Klebsiella pneumoniae, Thermoanaerobacterium thermosaccharolyticum, Corynebacterium glutamicum and Saccharomyces cerevisiae. Most preferably, the microorganism of the invention is either the species Escherichia coli or Corynebacterium glutamicum.

The term “recombinant microorganism”, “genetically modified microorganism”, or “genetically engineered microorganism”, as used herein, refers to a microorganism as defined above that is not found in nature and therefore genetically differs from its natural counterpart. In other words, it refers to a microorganism that is modified by introduction and/or by deletion and/or by modification of its genetic elements. Such modification can be performed by genetic engineering, by forcing the development and evolution of new metabolic pathways by culturing the microorganism under specific selection pressure, or by combining both methods (see, e.g. WO2005/073364 or WO2008/116852).

A microorganism genetically modified for the production of methionine according to the invention therefore means that said microorganism is a recombinant microorganism as defined above that is capable of producing methionine. In other words, said microorganism has been genetically modified to allow higher productions of methionine than the non-modified microorganism.

According to the invention, the amount of methionine produced by the recombinant microorganism of the invention, and particularly the methionine yield (ratio of methionine produced per carbon source, in gram/gram or mol/mol), is higher in the modified microorganism compared to the corresponding unmodified microorganism.

In the context of the invention, “non-modified microorganism” and “unmodified microorganism” means a microorganism which does not contain any genetic modification of gene(s) involved in methionine production.

The modified microorganisms of the invention are optimized for methionine production and further genetically modified for expressing functional genes encoding a thiocarboxylated protein, a polypeptide having an homoserine O-acetyltransferase activity without feedback inhibition by methionine and/or S-adenosylmethionine and a polypeptide having O-acetylhomoserine sulfhydrylase activity. The terms “genetically modified microorganism producing methionine” or “methionine-producing microorganism” or “microorganism genetically modified for the production of methionine” or “microorganism optimized for the production of methionine” or “recombinant L-methionine producing strain” and expression derived thereof designate a microorganism as defined above producing higher levels of methionine than the non-modified microorganism. Microorganisms optimized for methionine production are well known in the art, and have been disclosed in particular in patent applications WO2005/111202, WO2007/077041, WO2009/043803, WO2010/020681, WO2011/073738, WO2011/080542, WO2011/080301, WO2012/055798, WO2013/001055, WO2013/190343, WO2015/028675 and WO2015/028674. For the sake of clarity the genetically modified microorganism producing methionine and expressing the thiocarboxylated protein, the homoserine O-acetyltransferase and the O-acetylhomoserine sulfhydrylase is named in this disclosure “recombinant microorganism of the invention” or “microorganism of the invention” and expression derived thereof.

As further explained below, the genetically modified microorganism producing methionine and the microorganism of the invention can be genetically modified by modulating the expression level of one or more endogenous genes, and/or by expressing one or more heterologous genes in said microorganism.

By “modulating”, it is meant herein that the expression level of said gene is up-regulated, downregulated, or even completely abolished by comparison to its natural expression level. Such modulation can therefore result in an enhancement of the activity of the gene product, or alternatively, in a lower or null activity of the endogenous gene product.

By “gene”, it is meant herein a nucleic acid molecule or polynucleotide that codes for a particular protein (i.e. polypeptide), or in certain cases, for a functional or structural RNA molecule. In the context of the present invention, the genes referred herein encode proteins, such as enzymes.

The term “functional gene” means that the expression of the gene is functional that is to say that the nucleotidic sequence contains all elements allowing gene transcription and gene translation and potentially excretion of the protein encoded by said gene.

The terms “encoding” or “coding” refer to the process by which a polynucleotide, (i.e. a gene), through the mechanisms of transcription and translation, produces an amino-acid sequence. Genes according to the invention are either endogenous genes or exogenous.

The term “endogenous gene” refers herein to a gene that is naturally present in the microorganism.

An endogenous gene can be overexpressed by introducing heterologous sequences which favour upregulation in addition to endogenous regulatory elements or by substituting those endogenous regulatory elements with such heterologous sequences, or by introducing one or more supplementary copies of the endogenous gene into the chromosome or a plasmid within the microorganism. Endogenous gene activity and/or expression level can also be modified by introducing mutations into their coding sequence to modify the gene product. A deletion of an endogenous gene can also be performed to inhibit totally its expression within the microorganism. Another way to modulate the expression of an endogenous gene is to exchange its promoter (i.e. wild type promoter) with a stronger or weaker promoter to up or down regulate the expression level of this gene. Promoters suitable for such purpose can be homologous or heterologous and are well-known in the art. It is within the skill of the person in the art to select appropriate promoters for modulating the expression of an endogenous gene.

In addition, or alternatively, a microorganism can be genetically modified to express one or more exogenous genes, provided that said genes are introduced into the microorganism with all the regulatory elements necessary for their expression in the host microorganism. The modification or “transformation” of microorganisms with exogenous DNA is a routine task for those skilled in the art.

By “exogenous gene” or “heterologous gene”, it is meant herein that said gene is not naturally occurring in the microorganism. In order to express an exogenous gene in a microorganism, such gene can be directly integrated into the microorganism chromosome, or be expressed extra-chromosomally by plasmids or vectors within the microorganism. A variety of plasmids, which differ in respect of their origin of replication and of their copy number in a cell, are well known in the art and can be easily selected by the skilled practitioner for such purpose. Exogenous genes according to the invention are advantageously homologous genes.

In the context of the invention, the term “homologous gene” or “homolog” not only refers to a gene inherited by two species (i.e. microorganism species) by a theoretical common genetic ancestor, but also includes genes which may be genetically unrelated that have, nonetheless, evolved to encode proteins which perform similar functions and/or have similar structure (i.e. functional homolog). Therefore the term “functional homolog” refers herein to a gene that encodes a functionally homologous protein.

Using the information available in databases such as Uniprot (for proteins), Genbank (for genes), or NCBI (for proteins or genes), those skilled in the art can easily determine the sequence of a specific protein and/or gene of a microorganism, and identify based on this sequence the one of equivalent genes, or homologs, in another microorganism. This routine work can be performed by a sequence alignment of a specific gene sequence of a microorganism with gene sequences or the genome of other microorganisms, which can be found in the above mentioned databases. Such sequence alignment can advantageously be performed using the BLAST algorithm developed by Altschul et al. (1990). Once a sequence homology has been established between those sequences, a consensus sequence can be derived and used to design degenerate probes in order to clone the corresponding homolog gene of the related microorganism. These routine methods of molecular biology are well known to those skilled in the art.

It shall be further understood that, in the context of the present invention, should an exogenous gene encoding a protein of interest be expressed in a specific microorganism, a synthetic version of this gene is preferably constructed by replacing non-preferred codons or less preferred codons with preferred codons of said microorganism which encode the same amino acid. It is indeed well-known in the art that codon usage varies between microorganism species, which may impact the expression level of the protein of interest. To overcome this issue, codon optimization methods have been developed, and are extensively described by Graf et al. (2000), Deml et al. (2001) and Davis & Olsen (2011). Several software have notably been developed for codon optimization determination such as the GeneOptimizer® software (Lifetechnologies) or the OptimumGene™ software of GenScript. In other words, the exogenous gene encoding a protein of interest is preferably codon-optimized for expression in a specific microorganism.

The microorganism according to the invention can also be genetically modified to increase or decrease the expression of one or more genes.

The term “decrease the expression” or “attenuation of expression” according to the invention denotes the partial or complete suppression of the expression of the corresponding gene, which is then said to be ‘decreased’ or ‘attenuated’. This suppression of expression can be either an inhibition of the expression of the gene, a deletion of all or part of the promoter region necessary for the gene expression, a deletion of all or part of the coding region of the gene, or the exchange of the wild type promoter by a weaker natural or synthetic promoter or by an inducible promoter. The man skilled in the art knows a variety of promoters which exhibit different strength and which promoter to use for a weak or an inducible genetic expression. Preferentially, the attenuation of a gene is essentially the complete deletion of that gene, which can be replaced by a selection marker gene that facilitates the identification, isolation and purification of the strains according to the invention. A gene is inactivated preferentially by the technique of homologous recombination (Datsenko & Wanner, 2000).

The terms “increased expression”, “enhanced expression” or “overexpression” and grammatical equivalents thereof, are used interchangeably in the text and have a similar meaning. These terms mean that the expression of an endogenous or exogenous gene or the production of an enzyme or a protein is increased compared to the non modified microorganism leading to an increase in the intracellular concentration of a ribonucleic acid, a protein or an enzyme compared to the non modified microorganism. The man skilled in the art knows different means and methods to measure ribonucleic acid concentration or protein concentration in the cell including for instance use of Reverse Transcription Polymerase Chain Reaction (RT-PCR) to determine ribonucleic acid concentration and use of specific antibody to determine concentration of specific protein.

Increase production of a protein or an enzyme is obtained by increasing expression of the gene encoding said protein or enzyme by several techniques well known by the man skilled in the art.

In the context of the present invention, the terms “overexpress”, “overexpression” or “overexpressing” could be used to designate an increase in transcription of a gene in a microorganism.

Increasing the transcription of a gene, whether endogenous or exogenous, can be achieved by increasing the number of its copies within the microorganism and/or by using a promoter leading to a higher level of expression of the gene compared to the wild type promoter.

As indicated above, to increase the number of copies of a gene in the microorganism, said gene can be encoded chromosomally or extra-chromosomally. When the gene of interest is to be encoded on the chromosome, several copies of the gene can be introduced on the chromosome by methods of genetic recombination, which are well-known to in the art (e.g. gene replacement). When the gene is to be encoded extra-chromosomally in the microorganism, it can be carried by different types of plasmid that differ in respect to their origin of replication depending on the microorganism in which they can replicate, and by their copy number in the cell. The microorganism transformed by said plasmid can contain 1 to 5 copies of the plasmid, or about 20 copies of it, or even up to 500 copies of it, depending on the nature of the plasmid. Examples of low copy number plasmids which can replicate in E. coli include, without limitation, the pSC101 plasmid (tight replication), the RK2 plasmid (tight replication), as well as the pACYC and pRSF1010 plasmids, while an example of high copy number plasmid which can replicate in E. coli is pSK bluescript II.

Promoters which can increase the expression level of a gene are also well-known to the skilled person in the art, and can be homologous (originating from same species) or heterologous (originating from a different species or artificial promoter). Examples of such promoters include, without limitation, the promoters Ptrc, Ptac, Plac, and P_{R} and P_{L} of the lambda phage. These promoters can also be induced (“inducible promoters”) by a particular compound or by specific external condition like temperature or light.

The terms “overproduce”, “overproduction” or “overproducing” could also be used to designate an increase in the translation of a mRNA in a microorganism.

Increasing translation of the mRNA can be achieved by modifying the Ribosome Binding Site (RBS). A RBS is a sequence on mRNA that is bound by the ribosome when initiating protein translation. It can be either the 5′ cap of a mRNA in eukaryotes, a region 6-7 nucleotides upstream of the start codon AUG in prokaryotes (called the Shine-Dalgarno sequence), or an internal ribosome entry site (IRES) in viruses. By modifying this sequence, it is possible to change the protein translation initiation rate, to proportionally alter its production rate, and control its level activity inside the cell. It is also possible to optimize the strength of a RBS sequence to achieve a targeted translation initiation rate by using the software RBS CALCULATOR (Salis, 2011). It is within the skill of the person in the art to select the RBS sequence based on the nature of the mRNA.

The term “activity” of an enzyme is used interchangeably with the term “function” and designates, in the context of the invention, the reaction that is catalyzed by the enzyme. The man skilled in the art knows how to measure the enzymatic activity of said enzyme.

The term “increased activity” or “enhanced activity” designates an enzymatic activity that is superior to the enzymatic activity of the non modified microorganism. Increasing such activity can be obtained by improving the protein catalytic efficiency, by decreasing protein turnover, by decreasing messenger RNA (mRNA) turnover in addition to the techniques described above for increasing transcription of the gene encoding protein or enzyme, or increasing translation of the mRNA.

Improving the protein catalytic efficiency means increasing the kcat and/or decreasing the Km for a given substrate and/or a given cofactor, and/or increasing the Ki for a given inhibitor. kcat, Km and Ki are Michaelis-Menten constants that the man skilled in the art is able to determine (Segel, 1993). Decreasing protein turnover means stabilizing the protein. Methods to improve protein catalytic efficiency and/or decrease protein turnover are well known from the man skilled in the art. Those include rational engineering with sequence and/or structural analysis and directed mutagenesis, as well as random mutagenesis and screening. Mutations can be introduced by site-directed mutagenesis by conventional methods such as Polymerase Chain Reaction (PCR), by random mutagenesis techniques, for example via mutagenic agents (Ultra-Violet rays or chemical agents like nitrosoguanidine (NTG) or ethylmethanesulfonate (EMS)) or DNA shuffling or error-prone PCR. Stabilizing the protein can also be achieved by adding a “tag” peptide sequence either at the N-terminus or the C-terminus of the protein. Such tags are well known in the art, and include, among others, the Glutathione-S-Transferase (GST).

Decreasing mRNA turnover can be achieved by modifying the gene sequence of the 5′-untranslated region (5′-UTR) and/or the coding region, and/or the 3′-UTR (Carrier and Keasling, 1999).

The terms “attenuated activity” or “reduced activity” of an enzyme mean either a reduced specific catalytic activity of the protein obtained by mutation in the aminoacids sequence and/or decreased concentrations of the protein in the cell obtained by mutation of the nucleotidic sequence or by deletion of the coding region of the gene as described above.

Decreasing the activity of a protein can mean either decreasing its specific catalytic activity and/or decreasing expression of the corresponding gene in the cell by way of mutation, suppression, insertion or modification of single or multiple residues in a polynucleotide leading to alterations arising within a protein-encoding region of a gene as well as alterations in regions outside of a protein-encoding sequence such as, but not limited to, regulatory or promoter sequences. The alteration may be a mutation of any type and for instance: a point mutation, a frame-shift mutation, a nonsense mutation, an insertion or a deletion of part or all of a gene as described above.

The terms “feedback sensitivity” or “feedback inhibition” refer to a cellular mechanism control in which one or several enzymes that catalyse the production of a particular substance in the cell are inhibited or less active when that substance has accumulated to a certain level. So the terms “reduced feedback sensitivity” or “reduced feedback inhibition” or “without feedback inhibition” mean that the activity of such a mechanism is decreased or suppressed compared to a non modified microorganism. The man skilled in the art knows how to modify the enzyme to obtain this result. Such modifications have been described in the patent application WO 2005/111202 or in the U.S. Pat. No. 7,611,873.

The present invention is directed to a genetically modified microorganism producing methionine by fermentation, wherein said microorganism is further genetically modified for expressing functional genes encoding:
a thiocarboxylated protein,
a polypeptide having an homoserine O-acetyltransferase activity without feedback inhibition by methionine and/or S-adenosylmethionine and,
a polypeptide having O-acetylhomoserine sulfhydrylase activity.

In a first aspect of the invention the recombinant microorganism of the invention expresses functional genes encoding a thiocarboxylated protein as sulfur donor in the methionine biosynthetic pathway.

Thiocarboxylated proteins or protein thiocarboxylates are proteins wherein the carboxylic acid function at C-terminal position has one or both of the oxygen replaced by sulfur (R—COSH, R—CSOH, R—CSSH). These proteins are important intermediates in a variety of biochemical sulfide transfer reactions. These proteins are members of a growing family of biosynthetic sulfide donors and are involved in a variety of biosynthetic pathways.

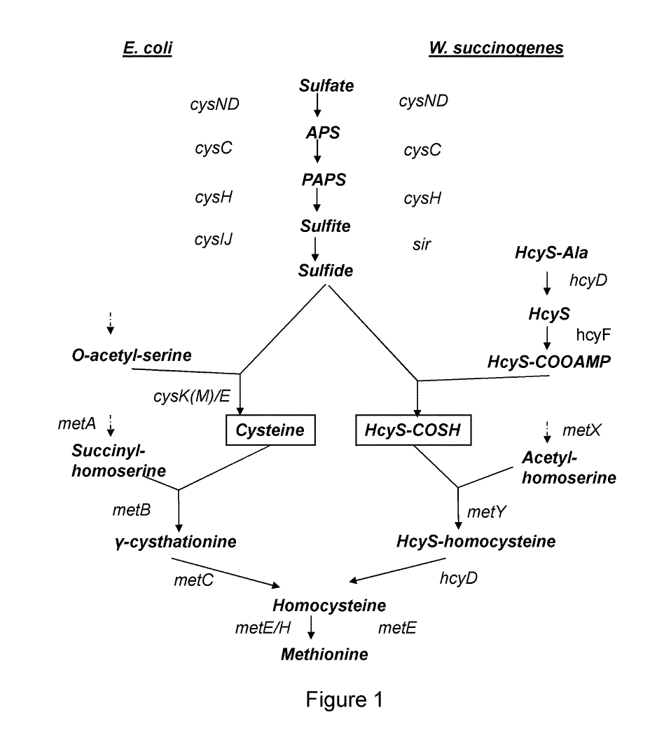

In the methionine biosynthetic pathway the thiocarboxylated protein reacts with acetylhomoserine to form the complex thiocarboxylated protein-homocysteine by the action of O-acetylhomoserine sulfhydrylase. Then the complex is hydrolysed to liberate homocysteine finally methylated to form methionine as described in FIG. 1.

In one embodiment the functional genes encoding the thiocarboxylated protein expressed in the microorganism of the invention are endogenous or heterologous.

Preferably, the functional genes encoding the thiocarboxylated protein expressed in the microorganism of the invention are heterologous.

Most preferably the recombinant microorganism of the invention overexpresses the genes hcyS, hcyD, hcyF and sir from Wolinella succinogenes and encoding the thiocarboxylated protein HcyS. The hcyS gene as set forth in SEQ ID NO: 1 encodes the protein HcyS-Ala as set forth in SEQ ID NO: 2. The hcyD gene as set forth in SEQ ID NO: 3 encodes a metalloprotease as set forth in SEQ ID NO: 4 involved in C-terminal processing of HcyS-Ala by removing the C-terminal alanine from HcyS-Ala for giving HcyS protein but also for an enzyme catalyzing the release of homocysteine from HcyS-homocysteine. The hcyF gene as set forth in SEQ ID NO: 5 encodes an enzyme as set forth in SEQ ID NO: 6 catalyzing the adenylation of HcyS protein. HcyS acyl-adenylate then undergoes nucleophilic substitution by bisulfide produced by the protein Sir as set forth SEQ ID NO: 7 encoded by the sir gene as set forth in SEQ ID NO: 8.

In a second aspect of the invention the recombinant microorganism of the invention expresses functional genes encoding for a polypeptide having an homoserine O-acetyltransferase activity without feedback inhibition by methionine and/or S-adenosylmethionine. This enzyme activity allows the cell to accumulate O-acetylhomoserine able to react with the thiocarboxylated protein described above.

A polypeptide having an homoserine O-acetyltransferase activity is a polypeptide having an enzyme activity catalyzing the chemical reaction:

acetyl-CoA+L-homoserine [symbol] CoA+O-acetyl-L-homoserine

Thus the two substrates of this enzyme are acetyl-CoA and L-homoserine, whereas its two products are CoA and O-acetyl-L-homoserine.

This enzyme belongs to the family of transferases (EC 2 enzyme), specifically those acyltransferases transferring groups other than aminoacyl groups (EC 2.3 enzyme). The systematic name of this enzyme class is acetyl-CoA:L-homoserine O-acetyltransferase. Other names in common use include homoserine acetyltransferase, homoserine transacetylase, homoserine-O-transacetylase, and L-homoserine O-acetyltransferase or more currently MetX protein. This enzyme participates in methionine metabolism and sulfur metabolism.

The homoserine O-acetyltransferase enzyme activity may be controlled by a feedback inhibition mechanism with methionine and/or S-adenosylmethionine or not that is to say that feedback inhibition by methionine and/or S-adenosylmethionine is reduced or suppressed.

The enzyme having homoserine O-acetyltransferase activity to be present in the recombinant microorganism of the invention has no feedback inhibition by methionine and/or S-adenosylmethionine, the activity of said enzyme being not inhibited by methionine and/or S-adenosymethionine: the O-acetylhomoserine formation pool is not suppressed or decreased by a methionine and/or S-adenosylmethionine concentration level.

In another embodiment of the invention the gene metX encoding the enzyme having homoserine O-acetyltransferase activity is endogenous or heterologous.

Preferably, the gene encoding the enzyme having homoserine O-acetyltransferase activity is heterologous and may originate from a variety of microorganisms. Microorganisms from which a gene metX encoding an enzyme having homoserine O-acetyltransferase activity can be obtained include Corynebacterium species, Leptospira species, Deinococcus species, Pseudomonas species or Mycobacterium species but are not limited thereto. Preferably the enzyme having homoserine O-acetyltransferase activity may be encoded by a gene metX originating from a strain selected from a group consisting of Corynebacterium glutamicum, Leptospira meyerei, Deinococcus radiodurans, Pseudomonas aeruginosa and Mycobacterium smegmatis. The metX gene as set forth in SEQ ID NO: 9 originating from Leptospira meyeri encodes an enzyme having an homoserine O-acetyltransferase activity without feedback inhibition by methionine and/or S-adenosylmethionine as set forth in SEQ ID NO: 10 (Bourhy et al, 1997). Other homoserine O-acetyltransferases showing resistance to feedback inhibition can be obtained by techniques well known by the man skilled in the art and are notably described in WO2005111202, U.S. Pat. No. 8,551,742, EP2290051 and WO2008013432 patent applications.

Most preferably the recombinant microorganism of the invention expresses the gene metX from Leptospira meyeri encoding an enzyme having an homoserine O-acetyltransferase activity without feedback inhibition by methionine and/or S-adenosylmethionine and even most preferably said gene is overexpressed.

In a third aspect of the invention the recombinant microorganism of the invention expresses functional genes encoding a polypeptide having O-acetylhomoserine sulfhydrylase activity.

This enzyme catalyzes the reaction of thiocarboxylated protein HcyS with O-acetylhomoserine to form the complex HcyS-Homocysteine and allows incorporation of the sulfur group from thiocarboxylated protein HcyS to acetylhomoserine.

This enzyme belongs to the family of transferases (EC 2 enzyme) that transfer specific functional group (e.g. a methyl or glycosyl group) from one molecule (called the donor) to another (called the acceptor). Specifically the enzyme transfers alkyl or aryl groups, other than methyl groups (EC 2.5.1). The specific name of this enzyme is O-acetyl-L-homoserine:methanethiol 3-amino-3-carboxypropyltransferase. Other names in common use include O-acetyl-L-homoserine acetate-lyase (adding methanethiol), O-acetyl-L-homoserine sulfhydrolase, O-acetylhomoserine (thiol)-lyase, O-acetylhomoserine sulfhydrolase and methionine synthase or more currently MetY protein. This enzyme participates in methionine metabolism and sulfur metabolism.

In one embodiment the gene metY encoding the enzyme having O-acetylhomoserine sulfhydrylase activity is endogenous or heterologous.

Preferably, the gene encoding the enzyme having O-acetylhomoserine sulfhydrylase activity is heterologous.

Most preferably the microorganism of the invention expresses the gene metY from Wolinella succinogenes as set forth in SEQ ID NO: 11 encoding a polypeptide having O-acetylhomoserine sulfhydrylase activity as set forth in SEQ ID NO: 12, and even most preferably said gene is overexpressed. Said overexpression may also be optimized by expressing a modified metY gene from Wolinella succinogenes as set forth in SEQ ID NO: 13, thus encoding a polypeptide having O-acetylhomoserine sulfhydrylase activity as set forth in SEQ ID NO: 14.

Preferably, in the recombinant microorganism of the invention, the gene encoding a polypeptide having homoserine O-acetyltransferase activity and the gene encoding a polypeptide having O-acetylhomoserine sulfhydrylase activity are heterologous.

Preferably the microorganism used in the invention is able to produce the L-methionine amino acid. More preferably the genetically modified microorganism producing methionine of the invention is optimized for the production of L-methionine.

Genes involved in methionine production in a microorganism are well known in the art, and comprise genes involved in the methionine specific biosynthesis pathway as well as genes involved in precursor-providing pathways and genes involved in methionine consuming pathways.

Efficient production of methionine requires the optimisation of the methionine specific pathway and several precursor-providing pathways. L-Methionine producing strains have been described in patent applications WO2005/111202, WO2007/077041 and WO2009/043803, WO2010/020681, WO2011/073738, WO2011/080542, WO2011/080301, WO2012/055798, WO2013/001055, WO2013/190343, WO2015/028675 and WO2015/028674 which are incorporated as reference into this application.

For improving the production of L-methionine, the microorganism genetically modified for the production of methionine may exhibit:
an increased expression of at least one gene selected in the group consisting of:
   cysP which encodes a periplasmic sulfate binding protein, as described in WO2007/077041 and in WO2009/043803,
   cysU which encodes a component of sulfate ABC transporter, as described in WO2007/077041 and in WO2009/043803,
   cysW which encodes a membrane bound sulfate transport protein, as described in WO2007/077041 and in WO2009/043803,
   cysA which encodes a sulfate permease, as described in WO2007/077041 and in WO2009/043803,
   cysM which encodes an O-acetyl serine sulfhydralase, as described in WO2007/077041 and in WO2009/043803,
   cysI and cysJ encoded respectively the alpha and beta subunits of a sulfite reductase as described in WO2007/077041 and in WO2009/043803. Preferably cysI and cysJ are overexpressed together,
   cysH which encodes an adenylylsulfate reductase, as described in WO2007/077041 and in WO2009/043803.

Increasing C1 metabolism is also a modification that leads to improved methionine production. It relates to the increase of the activity of at least one enzyme involved in the C1 metabolism chosen among GcvTHP, Lpd, MetF or MetH. In a preferred embodiment of the invention, the one carbon metabolism is increased by enhancing the expression and/or the activity of at least one of the following:
gcvT, gcvH, gcvP, and lpd, coding for the glycine cleavage complex, as described in patent application WO 2007/077041. The glycine-cleavage complex (GCV) is a multienzyme complex that catalyzes the oxidation of glycine, yielding carbon dioxide, ammonia, methylene-THF and a reduced pyridine nucleotide. The GCV complex consists of four protein components, the glycine dehydrogenase said P-protein (GcvP), the lipoyl-GcvH-protein said H-protein (GcvH), the aminomethyltransferase said T-protein (GcvT), and the dihydrolipoamide dehydrogenase said L-protein (GcvL or Lpd). P-protein catalyzes the pyridoxal phosphate-dependent liberation of CO2 from glycine, leaving a methylamine moiety. The methylamine moiety is transferred to the lipoic acid group of the H-protein, which is bound to the P-protein prior to decarboxylation of glycine. The T-protein catalyzes the release of NH3 from the methylamine group and transfers the remaining C1 unit to THF, forming methylene-THF. The L protein then oxidizes the lipoic acid component of the H-protein and transfers the electrons to NAD⁺, forming NADH;
MetF encoding a methylenetetrahydrofolate reductase, as described in patent application WO2007/07704. In a specific embodiment of the invention, the activity of MetF is enhanced by overexpressing the gene metF and/or by optimizing the translation. In another specific embodiment of the invention, overexpression of metF gene is achieved by expressing the gene under the control of a strong promoter belonging to the Ptrc family promoters, or under the control of an inducible promoter, like a temperature inducible promoter P_{R} as described in application WO2011/073738. According to another embodiment of the invention, optimisation of the translation of the protein MetF is achieved by using a RNA stabiliser. Other means for the overexpression of a gene are known to the expert in the field and may be used for the overexpression of the metF gene.

The overexpression of at least one of the following genes involved in serine biosynthesis also reduces the production of by-product isoleucine:
serA which encodes a phosphoglycerate dehydrogenase, as described in WO2007/077041 and in WO2009/043803,
serB which encodes a phosphoserine phosphatase, as described in WO2007/077041 and in WO2009/043803,
serC which encodes a phosphoserine aminotransferase, as described in WO2007/077041 and in WO2009/043803.

The overexpression of the following genes has already been shown to improve the production of methionine:
thrA or thrA alleles which encode aspartokinases/homoserine dehydrogenase with reduced feed-back inhibition to threonine (thrA*), as described in WO2009/043803 and WO2005/111202,
metL encoding for a polypeptide having bifunctionnal aspartokinase/homoserine dehydrogenase.
ptsG which encodes the glucose-specific phosphoenolpyruvate (PEP) phosphotransferase system (PTS) permease, as described in WO 2013/001055,
metH which encodes a cobalamin-dependent methionine synthase, as described in WO2015/028674,
metE which encodes a cobalamin-independent methionine synthase, as described in WO2013/190343,
fldA which encodes an essential flavodoxin containing FMN as a prosthetic group which interacts with Fpr and MetH proteins, as described in WO2015/028674,
fpr which encodes a flavodoxin NADP+ reductase required for the activation of the methionine synthase MetH as described in WO2015/028674. The reductase uses non covalently bound FAD as a cofactor,
pntAB operon which encodes respectively the α-subunit and the an inner membrane protein with nine predicted transmembrane domains of the membrane bound proton translocating pyridine nucleotide transhydrogenase, as described in WO 2012/055798,
ygaZH operon which encodes a member of the branched chain amino acid exporter (LIV-E) family responsible for export of L-valine and L-methionine,
pyc which encodes pyruvate carboxylase as described in patent application WO 2012/055798. Increasing activity of pyruvate carboxylase is obtained by overexpressing the corresponding gene or modifying the nucleic sequence of this gene to express an enzyme with improved activity. In another embodiment of the invention, the pyc gene is introduced on the chromosome in one or several copies by recombination or carried by a plasmid present at least at one copy in the modified microorganism. The pyc gene originates from Rhizobium etli, Bacillus subtilis, Pseudomonas fluorescens, Lactococcus lactis or Corynebacterium species. In a preferred embodiment, the microorganism of the invention overexpresses pyc gene from Rhizobium etli.
and/or an decrease of the expression of at least one of the following genes:
pykA which encodes a pyruvate kinase, as described in WO2007/077041 and in WO2009/043803,
pykF which encodes a pyruvate kinase, as described in WO2007/077041 and in WO2009/043803,
purU which encodes a formyltetrahydrofolate deformylase, as described in WO2007/077041 and in WO2009/043803,
yncA which encodes a N-acetyltransferase, as described in WO 2010/020681,
metJ which encodes a repressor of the methionine biosynthesis pathway, as described in WO2005/111202. The repressor protein MetJ is responsible for the down-regulation of the methionine regulon as was suggested in patent application JP2000/157267,
udhA which encodes a soluble pyridine nucleotide transhydrogenase which catalyses essentially the oxidation of NADPH into NADP⁺ via the reduction of NAD⁺ into NADH as described in patent application WO 2012/055798.
dgsA which encodes a transcriptional dual regulator that controls the expression of a number of genes encoding enzymes of the phosphotransferase (PTS) and phosphoenolpyruvate (PEP) systems, as described in WO 2013/001055,
sgrS which encodes a small RNA which regulates post-transcriptionally the abundance of PtsG, as described in WO 2013/001055
sgrT which encodes a regulator which plays a role in the glucose-phosphate stress response, regulating the activity of PtsG, as described in WO 2013/001055
metNIQ operon which encodes for the subunits of the ABC transporter involved in the uptake of methionine.

Genes may be expressed under control of an inducible promoter. Patent application WO2011/073738 describes a L-methionine producing strain that expresses a thrA allele with reduced feed-back inhibition to threonine under the control of an inducible promoter (thrA*). This application is incorporated as reference into this application. In a specific embodiment of the invention, the thrA or thrA allele, pyc, pntAB, ygaZH or ptsG genes are under control of a temperature inducible promoter. In a most preferred embodiment, the temperature inducible promoter used belongs to the family of P_{R} or P_{L} promoters.

In a particular embodiment of the invention, the overexpressed genes are at their native position on the chromosome or are integrated at a non-native position. For an optimal L-methionine production, several copies of the gene may be required, and these multiple copies are integrated into specific loci, whose modification does not have a negative impact on methionine production.

Examples for locus into which a gene may be integrated, without disturbing the metabolism of the cell, are disclosed in patent applications WO2011/073122, WO2011/073738 and WO2012/055798 which are incorporated by reference herein.

In one preferred embodiment of the invention the genetically modified microorganism producing methionine overexpresses at least one of the following genes: thrA or thrA allele encoding a polypeptide having aspartokinase/homoserine dehydrogenase activity with reduced feedback inhibition to threonine (thrA*), metL encoding a polypeptide having bifunctionnal aspartokinase/homoserine dehydrogenase, metE encoding a polypeptide having cobalamin-independent methionine synthase or metH encoding a polypeptide having cobalamin-dependent methionine synthase. More preferably the genetically modified microorganism producing methionine overexpresses the genes metH and metL. Even more preferably the genetically modified microorganism producing methionine overexpresses the genes thrA*, metH and metL.

In another preferred embodiment of the invention the metJ gene is deleted in order to avoid repression of methionine regulon. In this case neither the endogenous genes metL, metB, metE and/or metH, nor exogenous genes under the control of endogenous promoter belonging to the methionine regulon are repressed by MetJ protein.

In another preferred embodiment, the metJ gene is expressed but in this case the promoters of endogenous genes belonging to the MetJ regulon and the promoters used to control exogenous gene expression and which belong to the MetJ regulon are exchanged. Promoters suitable for such purpose can be homologous or heterologous and are well-known in the art.

In a particular aspect, the recombinant microorganism of the invention comprises the following genetic modifications:
the expression of the genes hcyS, hcyD, hcyF and sir from Wolinella succinogenes, metX from Leptospira meyeri and metY from Wolinella succinogenes are enhanced

In addition to these modifications, the recombinant microorganism of the invention preferably further comprises:
Increased expression of at least one the following genes: pyc, ptsG, pntAB, cysP, cysU, cysW, cysA, cysM, cysJ, cysI, cysH, gcvT, gcvH, gcvP, lpd, glyA, serA, serB, serC, metF, fldA, fpr, metN, metI, metQ, and/or
Attenuated expression of at least one of the following genes: metJ, pykA, pykF, purU, yncA, metE, dgsA, sgrS, sgrT, ygaZH or udhA.

More particularly, the recombinant microorganism of the invention comprises the following genetic modifications:
the expression of the genes hcyS, hcyD, hcyF and sir from Wolinella succinogenes, metX from Leptospira meyeri and metY from Wolinella succinogenes are enhanced
the expression of the genes cysPUWAM, cysJIH, thrA* and metH are enhanced.

Even more particularly, the recombinant microorganism of the invention comprises the following genetic modifications:
the expression of the genes hcyS, hcyD, hcyF and sir from Wolinella succinogenes, metX from Leptospira meyeri and metY from Wolinella succinogenes are enhanced
the expression of the genes cysPUWAM, cysJIH, gcvTHP, thrA*, metF and metH are enhanced, and,
the expression of the genes metJ is attenuated.

Or:
the expression of the genes hcyS, hcyD, hcyF and sir from Wolinella succinogenes, metX from Leptospira meyeri and metY from Wolinella succinogenes are enhanced
the expression of the genes cysPUWAM, cysJIH, gcvTHP, thrA*, metF and metH are enhanced, and,
the expression of the genes metJ, pykA, pykF and purU are attenuated.

Or:
the expression of the genes hcyS, hcyD, hcyF and sir from Wolinella succinogenes, metX from Leptospira meyeri and metY from Wolinella succinogenes are enhanced
the expression of the genes cysPUWAM, cysJIH, gcvTHP, thrA*, metF and metH are enhanced, and,
the expression of the genes pykA, pykF and purU are attenuated.
   The microorganism of the invention preferably belongs to the family of Enterobacteriaceae or Corynebacteriaceae, and most preferably is Escherichia coli.

Finally, the present invention is related to a method for the fermentative production of methionine comprising culturing a genetically modified microorganism producing methionine as described above and expressing functional genes encoding a thiocarboxylated protein, a polypeptide having an homoserine O-acetyltransferease activity without feedback inhibition by methionine and/or S-adenosylmethionine and a polypeptide having O-acetylhomoserine sulfhydrylase activity and recovering methionine from said culture medium.

According to a specific aspect of the invention, the method is performed with a recombinant microorganism overexpressing hcyS, hcyD, hcyF and sir genes from Wolinella succinogenes, metX gene from Leptospira meyeri and metY gene from Wolinella succinogenes.

According to another specific aspect of the method, the microorganism further overexpresses at least one of the following genes: thrA or thrA allele encoding a polypeptide having aspartokinase/homoserine dehydrogenase activity with reduced feedback inhibition to threonine (thrA*), metL encoding a polypeptide having bifunctionnal aspartokinase/homoserine dehydrogenase, metE encoding a polypeptide having cobalamin-independent methionine synthase or metH encoding a polypeptide having cobalamin-dependent methionine synthase.

According to the invention, the terms “fermentative process”, “culture” or “fermentation” are used interchangeably to denote the growth of a given microorganism on an appropriate culture medium containing a carbon source, a source of sulfur and a source of nitrogen. The growth is generally performed in fermenters with an appropriate growth medium adapted to the microorganism being used.

In the fermentative process of the invention, the source of carbon is used simultaneously for:
biomass production: growth of the microorganism by converting inter alia the carbon source of the medium, and,
methionine production: transformation of the same carbon source into methionine by the biomass.

The two steps are concomitant, and the transformation of the source of carbon by the microorganism to grow results in the L-methionine production in the medium, since the microorganism comprises a metabolic pathway allowing such conversion.

An “appropriate culture medium” means herein a medium (e.g., a sterile, liquid media) comprising nutrients essential or beneficial to the maintenance and/or growth of the microorganism such as carbon sources or carbon substrates; nitrogen sources, for example peptone, yeast extracts, meat extracts, malt extracts, urea, ammonium sulfate, ammonium chloride, ammonium nitrate and ammonium phosphate; phosphorus sources, for example monopotassium phosphate or dipotassium phosphate; trace elements (e.g., metal salts) for example magnesium salts, cobalt salts and/or manganese salts; as well as growth factors such as amino acids and vitamins.

The term “source of carbon” according to the invention denotes any source of carbon that can be used by those skilled in the art to support the normal growth of a microorganism, which can be hexoses such as glucose, galactose or lactose; pentoses; monosaccharides; disaccharides such as sucrose (molasses), cellobiose or maltose; oligosaccharides such as starch or its derivatives; hemicelluloses; glycerol and combinations thereof. An especially preferred carbon source is glucose. Another preferred carbon source is sucrose.

In a particular embodiment of the invention, the carbon source is derived from renewable feed-stock. Renewable feed-stock is defined as raw material required for certain industrial processes that can be regenerated within a brief delay and in sufficient amount to permit its transformation into the desired product. Vegetal biomass treated or not, is an interesting renewable carbon source.

The source of carbon is fermentable, i.e. it can be used for growth by microorganisms.

The term “source of sulfur” according to the invention refers to sulfate, thiosulfate, hydrogen sulfide, dithionate, dithionite, sulfite, methylmercaptan, dimethylsulfide, dimethyl disulfide and other methyl capped sulfides or a combination of the different sources. Preferred sulfur source in the culture medium is sulfate or thiosulfate or a mixture thereof. Another preferred sulfur source is dimethyl disulfide.

The term “source of nitrogen” corresponds to either an ammonium salt or ammoniac gas. Nitrogen comes from an inorganic (e.g., (NH₄)₂SO₄) or organic (e.g., urea or glutamate) source. In the invention sources of nitrogen in culture are (NH₄)₂HPO₄, (NH₄)₂S₂O₃ and NH₄OH.

The culture may be performed in such conditions that the microorganism is limited or starved for an inorganic substrate, in particular phosphate and/or potassium. Subjecting an organism to a limitation of an inorganic substrate defines a condition under which growth of the microorganisms is governed by the quantity of an inorganic chemical supplied that still permits weak growth. Starving a microorganism for an inorganic substrate defines the condition under which growth of the microorganism stops completely due, to the absence of the inorganic substrate.

Those skilled in the art are able to define the culture conditions and the composition of culture medium for the microorganisms according to the invention. In particular the bacteria are fermented at a temperature between 20° C. and 55° C., preferentially between 25° C. and 40° C., and more specifically about 30° C. for C. glutamicum and about 37° C. for E. coli.

As an example of known culture medium for E. coli, the culture medium can be of identical or similar composition to an M9 medium (Anderson, 1946, Proc. Natl. Acad. Sci. USA 32:120-128), an M63 medium (Miller, 1992; A Short Course in Bacterial Genetics: A Laboratory Manual and Handbook for Escherichia coli and Related Bacteria, Cold Spring Harbor Laboratory Press, Cold Spring Harbor, N.Y.) or a medium such as defined by Schaefer et al. (1999, Anal. Biochem. 270: 88-96).

As an example of known culture medium for C. glutamicum, the culture medium can be of identical or similar composition to BMCG medium (Liebl et al., 1989, Appl. Microbiol. Biotechnol. 32: 205-210) or to a medium such as described by Riedel et al. (2001, J. Mol. Microbiol. Biotechnol. 3: 573-583).

The method of the invention can be performed either in a batch process, in a fed-batch process or in a continuous process, and under aerobic, micro-aerobic or anaerobic conditions.

A fermentation “under aerobic conditions” means that oxygen is provided to the culture by dissolving gas into the liquid phase of the culture. This can be achieved by (1) sparging oxygen containing gas (e.g. air) into the liquid phase, or (2) shaking the vessel containing the culture medium in order to transfer the oxygen contained in the head space into the liquid phase. The main advantage of the fermentation under aerobic conditions is that the presence of oxygen as an electron acceptor improves the capacity of the strain to produce more energy under the form of ATP for cellular processes, thereby improving the general metabolism of the strain.

Micro-aerobic conditions can be used herein and are defined as culture conditions wherein low percentages of oxygen (e.g. using a mixture of gas containing between 0.1 and 10% of oxygen, completed to 100% with nitrogen) are dissolved into the liquid phase.

By contrast, “anaerobic conditions” are defined as culture conditions wherein no oxygen is provided to the culture medium. Strictly anaerobic conditions can be obtained by sparging an inert gas like nitrogen into the culture medium to remove traces of other gas. Nitrate can be used as an electron acceptor to improve ATP production by the strain and improve its metabolism.

In the invention, the fermentation is done in fed-batch mode. This refers to a type of fermentation in which supplementary growth medium is added during the fermentation, but no culture is removed until the end of the batch (except small volumes for samplings and HPLC/GCMS analysis). The process comprises two main steps; the first one which is a series of pre cultures in appropriate batch mineral medium and fed-batch mineral medium. Subsequently, a fermentor filled with appropriate minimal batch medium is used to run the culture with different fed-batch medium according to the desire production.

The method of the invention further comprises a step of recovering the methionine from the culture medium.

The action of “recovering methionine from the culture medium” designates the action of recovering methionine from the fermentation medium whatever its purity degree. “Recovering” means recovering the first product directly obtained from the fermentative process (fermentation must) which contains the product of interest (in this case methionine) and other co-products of the fermentation so with a more or less acceptable purity degree.

The “purifying” step consists of specifically purify the product of interest (in this case methionine) in order to obtain methionine with an improved purity degree.

Methionine might be recovered and purified by techniques and means well known by the man skilled in the art like distillation, ion-exchange chromatographic methods, precipitation, crystallisation or complexation with salts and particularly with calcium salts or ammonium salts.

The methods for the recovery and purification of the produced compounds are well known to those skilled in the art (see in particular WO 2005/007862, WO 2005/059155). Preferably, the step of recovering methionine comprises a step of concentration of methionine and/or its derivatives in the fermentation broth.

The amount of product in the fermentation medium can be determined using a number of methods known in the art, for example, high performance liquid chromatography (HPLC) or gas chromatography (GC). For example the quantity of methionine obtained in the medium is measured by HPLC after OPA/Fmoc derivatization using L-methionine (Sigma, Ref 64319) as a standard.

### Examples

The following experiments demonstrate how overexpression of genes encoding for proteins involved in production of the thiocarboxylated protein HcyS together with the overexpression of genes encoding for enzymes involved in production of acetyl-homoserine and HcyS-homocysteine in microorganisms such as E. coli improved methionine production.

In the examples given below, methods well known in the art were used to construct E. coli strains containing replicating vectors and/or various chromosomal insertions, deletions, and substitutions using homologous recombination well described by Datsenko & Wanner, (2000) for E. coli.

In the same manner, the use of plasmids or vectors to express or overexpress one or several genes in a recombinant microorganisms are well known by the man skilled in the art.

Examples of suitable E. coli expression vectors include pTrc, pACYC 184, pBR322, pUC18, pUC19, pKC30, pRep4, pHS1, pHS2, pPLc236 etc . . . .

### Protocols

Several protocols have been used to construct methionine producing strains described in the following examples.

Protocol 1 (Chromosomal modifications by homologous recombination, selection of recombinants and antibiotic cassette excision) and protocol 2 (Transduction of phage P1) used in this invention have been fully described in patent application WO2013/001055.

Protocol 3: Construction of Recombinant Plasmids

Recombinant DNA technology is well described and known by the man skilled in the art. Briefly, the DNA fragments are PCR amplified using oligonucleotides that the person skilled in the art is able to design and genomic DNA of the strain of interest is used as matrix. The DNA fragments and selected plasmid are digested with compatible restriction enzymes, ligated and then transformed in competent cells. Transformants are analysed and recombinant plasmids of interest are verified by DNA sequencing.

### Example 1: Overproduction of the Protein Thiocarboxylate-Dependent Methionine Biosynthesis Pathway of Wolinella succinogenes in a Recombinant L-Methionine Producing E. coli Strain—Strain 1, and Construction of Strains 2 and 3

Methionine producing strain used in this application: Strain 1 Strain 1: The L-methionine producing strain used as recipient for the overproduction of protein thiocarboxylate-dependent methionine biosynthesis pathway of Wolinella succinogenes is MG1655 metA*11 DmetJ Ptrc36-ARNmst17-metF Ptrc-metH PtrcF-cysJIH PtrcF-cysPUWAM PtrcO9-gcvTHP DpykA DpykF DpurU, described in the previous patent application WO2009/043803, and named strain 1 in this present patent application This strain is a L-methionine producing E. coli strain which doesn't possess fully optimization of its endogenous cysteine production pathway. This strain is used as reference for comparison with the recombinant strain of the invention which possesses protein thiocarboxylate-dependent methionine biosynthesis pathway of Wolinella succinogenes and which is described in this present application.

Overproduction of the Protein Thiocarboxylate-Dependent Methionine Biosynthesis Pathway of Wolinella succinogenes: Overexpression of hcyS, hcyF, hcyD and Sir from Wolinella succinogenes

The gene hcyS as set forth in SEQ ID NO: 1 encoding the sulfur transfer protein as set forth in SEQ ID NO: 2), the gene hcyD as set forth in SEQ ID NO: 3 encoding the metalloprotease as set forth in SEQ ID NO: 4, the gene hcyF as set forth in SEQ ID NO: 5 encoding the enzyme as set forth in SEQ ID NO: 6 that catalyses the adenylation of HcyS, and the gene sir as set forth in SEQ ID NO: 8 encoding the sulfite reductase as set forth in SEQ ID NO: 7, all from Wolinella succinogenes (ATCC29543D-5), were overexpressed in genetic background of strain 1.

The operon hcySFD-sir was overexpressed by using the same promoter as described for cysE gene into the pME101-thrA*1-cysE plasmid described in patent application WO2007/0770441, the ribosome binding site of each hcySFD-sir genes and the moderate plasmid copy number pCL1920 (Lerner & Inouye, 1990). More precisely, the hcySFD-sir operon, operatively linked to the chosen promoter, was cloned downstream of thrA*1 gene into the pME101-thrA*1 recombinant plasmid described in patent application WO2007/0770441. This plasmid was named pME1308.

Modification of the Recombinant L-Methionine Producing E. coli Strain with the Alternative Methionine Biosynthesis Pathway from Wolinella succinogenes: Deletion of metA*11 Allele and Overexpression of metX and metY Genes from Leptospira meyeri and Wolinella succinogenes, Respectively

To produce acetyl-homoserine instead of succinyl-homoserine with strain 1, the E. coli metA*11 gene coding for the homoserine O-succinyltransferase was deleted, and replaced by the overexpression of metX gene as set forth in SEQ ID NO: 9 from Leptospira meyeri (Leptospira meyeri serovar semaranga—DSM21536) encoding the homoserine O-acetyltransferase as set forth in SEQ ID NO: 10. Then, HcyS-homocysteine, which is the addition of acetyl-homoserine to HcyS thiocarboxylate (HcyS-COSH), is obtained by overexpressing the Wolinella succinogenes metY gene coding for the O-acetylhomoserine-sulfhydrylase.

### Deletion of metA*11 Allele of E. coli Strain 1—Construction of Strain 2

To delete metA*11 allele from strain 1, the homologous recombination strategy described by Datsenko & Wanner, 2000 (according to Protocol 1) was used. A fragment carrying a resistance marker, flanked by DNA sequence homologous to the sequences up- and downstream of metA*11 locus as set forth in SEQ ID NO: 15 and in SEQ ID NO: 16), was PCR amplified. The PCR product obtained was then introduced by electroporation into strain 1, in which the pKD46 vector had beforehand been introduced. The antibiotic resistant transformants were then selected and the deletion of the metA*11 gene associated to the resistance cassette was verified by a PCR analysis with appropriate oligonucleotides. The strain retained was designated strain 2.

Overexpression of metX of Leptospira meyeri

The metX gene of Leptospira meyeri was overexpressed by using the same promoter and ribosome binding site as described for metA*11 gene into the pCL1920-TTadc-CI857-PlambdaR*(−35)-thrA*1-cysE-PgapA-metA*11 plasmid described in the patent application WO2011/073122 (Example 1) and the moderate plasmid copy number pCL1920 (Lerner & Inouye, 1990). More precisely, the metX gene, operatively linked to the chosen promoter, was cloned downstream of the sir gene into the pME1308 recombinant plasmid described in this patent application. This plasmid was named pME1325.

Overexpression of metY of Wolinella succinogenes

The metY gene as set forth in SEQ ID NO: 11, encoding the protein METY as set forth in SEQ ID NO: 12) of Wolinella succinogenes (ATCC29543D-5) was overexpressed by using the promoter of E. coli metB gene (Kirby et al., 1986), the endogenous ribosome binding site of metY gene and the bacterial artificial chromosome (pCC1BAC, Epicentre). To optimize the overexpression, the star codon GTG of metY was also changed in ATG as set forth in SEQ ID NO: 13, thus encoding a methionine as the first amino acid instead of a valine as set forth in SEQ ID NO: 14). This plasmid was named pME1306.

Construction of Strain 3: Overproduction of the Protein Thiocarboxylate-Dependent Methionine Biosynthesis Pathway of Wolinella succinogenes in a Recombinant L-Methionine Producing E. coli Strain

The plasmids pME1325 and pME1306 were transformed into strain 2, giving rise to the strain 3.

### Example 2: Overproduction of MetX and MetY in a Recombinant L-Methionine Producing E. coli Strain Deleted for metA, and without the Alternative Methionine Biosynthesis Pathway from Wolinella succinogenes—Construction of Strain 4

The operon hcySFD-sir carried by the plasmid pME1325 was removed by using appropriate restriction enzymes. The resulting plasmid was named pME1338.

The plasmids pME1338 and pME1306 were transformed into strain 2, giving rise to the strain 4.

### Example 3: Growth and L-Methionine Production with the Alternative Methionine Biosynthesis Pathway from Wolinella succinogenes

Production strains were evaluated in small Erlenmeyer flasks. A 5.5 mL preculture was grown at 37° C. in a mixed medium (10% LB medium (Sigma 25%) with 2.5 g·L⁻¹ glucose and 90% minimal medium PC1). It was used to inoculate a 50 mL culture to an OD₆₀₀ of 0.2 in medium PC1. Spectinomycin and kanamycin were added at a concentration of 50 mg·L⁻¹ and ampicillin at 10 mg·L⁻¹ when it was necessary. The temperature of the cultures was 37° C. When the culture had reached an OD₆₀₀ of 5 to 7, extracellular amino acids were quantified by HPLC after OPA/Fmoc derivatization and other relevant metabolites were analyzed using HPLC with refractometric detection (organic acids and glucose) and GC-MS after silylation.

The methionine titer was expressed as followed:

$$Titer = \frac{methionine ( mol )}{volume ( L )}$$

TABLE 1
Minimal medium composition (PC1).

| Compound | Concentration (g · L⁻¹) |
|---|---|
| ZnSO₄•7H₂O | 0.0040 |
| CuCl₂•2H₂O | 0.0020 |
| MnSO₄•H₂O | 0.0200 |
| CoCl₂•6H₂O | 0.0080 |
| H₃BO₃ | 0.0010 |
| Na₂MoO₄•2H₂O | 0.0004 |
| MgSO₄•7H₂O | 1.00 |
| Citric acid | 6.00 |
| CaCl₂•2H₂O | 0.04 |
| K₂HPO₄ | 8.00 |
| Na₂HPO₄ | 2.00 |
| (NH₄)₂HPO₄ | 8.00 |
| NH₄Cl | 0.13 |
| NaOH 4M | Adjusted to pH 6.8 |
| FeSO_{4•}7H₂O | 0.04 |
| Thiamine | 0.01 |
| Glucose | 20.00 |
| Ammonium thiosulfate | 5.61 |
| Vitamin B12 | 0.01 |
| MOPS | 20.00 |
| IPTG | 0.0048 |

TABLE 2
Growth and L-methionine production levels (mM) for each
strain tested in Erlenmeyer flasks in minimal medium.

| Strain | Growth rate | Methionine production (mM) |
|---|---|---|
| Strain 1 | + | 1.7 |
| Strain 2 | − | 0 |
| Strain 4 | + | 1.1 |
| Strain 3 | ++ | 1.8 |

(−) means no growth at all (0 h⁻¹);
(+) corresponds to a growth rate between 0 and 0.2 h⁻¹;
(++) corresponds to a growth rate above 0.2 h⁻¹

The overexpression of the sulfur pathway from W. succinogenes in a recombinant L-methionine producing E. coli strain improves the growth rate and the L-methionine production (strain 3 compared to strain 4). This result shows that the alternative sulfur pathway functions in E. coli and improves the production of L-methionine. In strains 2, 3 and 4, the metA*1 gene was deleted to demonstrate the functionality of the protein thiocarboxylate dependent methionine biosynthetic pathway. However, equivalent strains with a functional copy of metA*11 gene on the chromosome were also constructed and evaluated in the same conditions. The results show that the metabolic pathway functions also in E. coli metA*11 strains (data not shown).

All this results demonstrate that the alternative sulfur pathway from Wolinella succinogenes functions in E. coli and improves the production of the sulfur containing amino acid L-methionine. Moreover the use of such pathway overcomes problems linked to cysteine overproduction encountered usually for this production.

### REFERENCES

Agren D., Schnell R., Oehlmann W., Singh M., Schneider G., 2008, Journal of Biological Chemistry, 283(46):31567-31574.
Altschul S, Gish W, Miller W, Myers E, Lipman D J, 1990, J. Mol. Biol, 215 (3): 403-410.
Anderson, 1946, Proc. Natl. Acad. Sci. USA., 32:120-128.
Bourhy P, Martel A, Margarita D, Saint-Girons I and Belfaiza J. 1997, Journal of Bacteriology, 179 (13): 4396-4398.
Carrier T & Keasling J. 1999, Biotechnol Prog., 15(1): 58-64.
Datsenko K. A., Wanner B. L., 2000, Proceedings of the National Academy of Sciences of the USA, 97:6640-6645
Davis J J & Olsen G J., 2011, Mol. Biol. Evol., 28(1):211-221.
Deml L, Bojak A, Steck S, Graf M, Wild J, Schirmbeck R, Wolf H, Wagner R., 2001, J. Virol., 75(22): 10991-11001.
Graf M, Bojak A, Deml L, Bieler K, Wolf H, Wagner R., 2000, J. Virol., 74(22): 10/22-10826.
Kirby T W., Hindenach B R., Greene R C., 1986, Journal of Bacteriology, 165(3):671-677
Krishnamoorthy K., Begley T P., 2011, Journal of the American Chemical Society, 133(2):379-386
Lerner C G, Inouye M., 1990, Nucleic Acids Res. 1990 Aug. 11; 18(15):4631.
Liebl W, Klamer R, Schleifer K H 1989, Appl. Microbiol. Biotechnol. 32: 205-210.
Miller, 1992 “A Short Course in Bacterial Genetics: A Laboratory Manual and Handbook for Escherichia coli and Related Bacteria”, Cold Spring Harbor Laboratory Press, Cold Spring Harbor, N.Y.
Riedel C, Rittmann D, Dangel P, Mickel B, Petersen S, Sahm H, Eikmanns B J., 2001, J Mol. Microbiol. Biotechnol. 3: 573-583.
Salis H, 2011, Methods Enzymol., 498:19-42.
Saunderson C. L., 1985, British Journal of Nutrition, 54:621-633
Schaefer U, Boos W, Takors R, Weuster-Botz D, 1999 Anal. Biochem. 270: 88-96.
Segel I H, 1993, Enzyme kinetics, John Wiley & Sons, pp. 44-54 and 100-112.
Taylor S V., Kelleher N L., Kinsland C., Chiu H J., Costello C A., Backstrom A D., McLafferty F W., Begley T P., 1998, Journal of Biological Chemistry, 273(26):16555-16560.

1-15. (canceled)

16. A genetically modified microorganism that produces methionine when fermented, wherein said microorganism comprises genetic modifications for expressing functional genes encoding:
a) a thiocarboxylated protein;
b) a polypeptide having an homoserine O-acetyltransferase activity without feedback inhibition by methionine and/or S-adenosylmethionine; and
c) a polypeptide having O-acetylhomoserine sulfhydrylase activity.

17. The microorganism of claim 16, wherein functional genes encoding a thiocarboxylated protein are heterologous.

18. The microorganism of claim 16, wherein the genes hcyS, hcyD, hcyF and sir from Wolinella succinogenes encoding a thiocarboxylated HcyS protein are overexpressed.

19. The microorganism of claim 16 wherein the gene encoding a polypeptide having homoserine O-acetyltransferase activity without feedback inhibition by methionine and/or S-adenosylmethionine and the gene encoding a polypeptide having O-acetylhomoserine sulfhydrylase activity are heterologous.

20. The microorganism of claim 16, wherein the gene encoding a polypeptide having homoserine O-acetyltransferase activity without feedback inhibition by methionine and/or S-adenosylmethionine is a metX gene from Leptospira meyeri.

21. The microorganism of claim 20, wherein the metX gene from Leptospira meyeri is overexpressed.

22. The microorganism of claim 16, wherein the gene encoding a polypeptide having O-acetylhomoserine sulfhydrylase activity is metY gene from Wolinella succinogenes.

23. The microorganism of claim 22 wherein a metY gene from Wolinella succinogenes is overexpressed.

24. The microorganism of claim 16, wherein said microorganism is further genetically modified to overexpress at least one of the following genes: thrA or a thrA allele encoding a polypeptide having aspartokinase/homoserine dehydrogenase activity with reduced feedback inhibition to threonine (thrA*), metL encoding a polypeptide having bifunctional aspartokinase/homoserine dehydrogenase, metE encoding a polypeptide having cobalamin-independent methionine synthase or metH encoding a polypeptide having cobalamin-dependent methionine synthase.

25. The microorganism of claim 16, wherein said microorganism comprises the following genetic modifications:
a) increased expression of at least one the following genes: pyc, ptsG, pntAB, cysP, cysU, cysW, cysA, cysM, cysJ, cysI, cysH, gcvT, gcvH, gcvP, lpd, glyA, serA, serB, serC, metF, fldA, fpr, metN, metI, metQ, and/or
b) attenuated expression of at least one of the following genes: metJ, pykA, pykF, purU, yncA, metE, dgsA, sgrS, sgrT, ygaZH or udhA.

26. The microorganism of claim 16, wherein said microorganism belongs to the family of Enterobacteriaceae or Corynebacteriaceae.

27. The microorganism of claim 26, wherein said Enterobacteriaceae bacterium is Escherichia coli.

28. The microorganism of claim 22, wherein the gene encoding a polypeptide having homoserine O-acetyltransferase activity without feedback inhibition by methionine and/or S-adenosylmethionine is a metX gene from Leptospira meyeri

29. The microorganism of claim 28, wherein the genes hcyS, hcyD, hcyF and sir from Wolinella succinogenes encoding a thiocarboxylated HcyS protein are overexpressed.

30. The microorganism of claim 29, wherein said microorganism comprises the following genetic modifications:
a) increased expression of at least one the following genes: pyc, ptsG, pntAB, cysP, cysU, cysW, cysA, cysM, cysJ, cysI, cysH, gcvT, gcvH, gcvP, lpd, glyA, serA, serB, serC, metF, fldA, fpr, metN, metI, metQ, and/or
b) attenuated expression of at least one of the following genes: metJ, pykA, pykF, purU, yncA, metE, dgsA, sgrS, sgrT, ygaZH or udhA.

31. The microorganism of claim 30, wherein said microorganism belongs to the family of Enterobacteriaceae or Corynebacteriaceae.

32. A method for the fermentative production of methionine, comprising:
a) culturing a genetically modified microorganism producing methionine expressing functional genes encoding a thiocarboxylated protein, a polypeptide having an homoserine O-acetyltransferease activity without feedback inhibition by methionine and/or S-adenosylmethionine and a polypeptide having O-acetylhomoserine sulfhydrylase activity and,
b) recovering methionine from said culture medium.

33. The method of claim 32, wherein said genetically modified microorganism overexpresses hcyS, hcyD, hcyF and sir genes from Wolinella succinogenes, metX gene from Leptospira meyeri and metY gene from Wolinella succinogenes.

34. The method of claim 32, wherein the genetically modified microorganism further overexpresses at least one of the following genes: thrA or thrA allele encoding a polypeptide having aspartokinase/homoserine dehydrogenase activity with reduced feedback inhibition to threonine (thrA*), metL encoding a polypeptide having bifunctionnal aspartokinase/homoserine dehydrogenase, metE encoding a polypeptide having cobalamin-independent methionine synthase or metH encoding a polypeptide having cobalamin-dependent methionine synthase.

35. The method of claim 34, wherein said genetically modified microorganism overexpresses hcyS, hcyD, hcyF and sir genes from Wolinella succinogenes, metX gene from Leptospira meyeri and metY gene from Wolinella succinogenes.
